(19) United States
(12) Patent Application Publication
(10) Pub. No.: US 2023/0204484 A1
(43) Pub. Date: Jun. 29, 2023

(54) DIAGNOSTIC SYSTEM AND METHOD FOR VALIDATING PERSONALIZED CELL THERAPY TREATMENTS

(71) Applicant: Bridging Biosciences, LLC, Frisco, TX (US)

(72) Inventors: Michael W. Jones, McKinney, TX (US); Tapley Holland, Frisco, TX (US)

(21) Appl. No.: 18/109,231

(22) Filed: Feb. 13, 2023

Related U.S. Application Data
(63) Continuation of application No. 16/138,873, filed on Sep. 21, 2018, now Pat. No. 10,712,249.
(60) Provisional application No. 62/561,537, filed on Sep. 21, 2017.

(51) Int. Cl. G01N 11/08

(52) U.S. Cl. CPC G01N 11/08

(57) ABSTRACT

This invention relates in general to the field of cell-therapy treatments and more particularly, but not by way of limitation, to systems and methods for validating personalized cell-therapy treatments. In various embodiments, the system may calculate an aspiration volume needed for centrifugation to achieve a concentrated target threshold dose for a particular cell therapy using various factors such as, for example, information about a patient and the efficiency of the concentration process. In various embodiments, the system may also provide an indication to a physician of relevant published cell therapies to assist the physician in administering a treatment protocol.

### CROSS-REFERENCE TO RELATED APPLICATIONS

This patent application is a continuation of U.S. Pat. Application No. 16/138,872, filed Sep. 21, 2018, entitled “Diagnostic System and Method for Validating Personalized Cell Therapy Treatments,” which claims priority to U.S. Prov. Pat. App. Ser. No. 62/561,537, filed Sep. 21, 2017, both of which are hereby incorporated by reference for all purposes.

### BACKGROUND

### Technical Field

This invention relates in general to the field of medical device data systems and more particularly, but not by way of limitation, to systems and methods for validating personalized cell therapies treatments.

### Background

The use of concentrated platelet rich plasma (PRP) or concentrated bone marrow aspirate (cBMA) is widely understood and its clinical benefits are well established for a variety of tissue applications. The definition of PRP is a volume of blood plasma that has been centrifuged in order to produce a finite volume of PRP concentrate (PRP concentrate contains enriched quantities of different growth factors, platelets or thrombocytes, cytokines, exosomes, white blood cells, etc.) in order to stimulate healing tissue and remodeling. The definition of cBMA is a volume of whole bone marrow that is centrifuged to produce a finite volume of cBMA.

For some therapeutic applications of PRP and cBMA, proven thresholds or doses have been established and verified. For example, it is generally agreed that an effective PRP concentration to stimulate angiogenesis is about 1.5×10⁶ platelets per mL. Angiogenesis (new blood vessel formation) is necessary for any type of tissue genesis. Furthermore, it has been observed that lower or higher concentrations of PRP were less effective, whereby excessively high concentrations had inhibitory effects. Various scientific and research settings have established recommended doses for antimicrobial capabilities, pain relief, and other treatment applications. In the last several years, dosing for many autologous therapeutic treatments and tissue regeneration applications have been established. However, known variabilities and a lack of translation from the research setting to the clinical setting has hampered the use and effectiveness of these treatments. This lack is encouraging physicians to continue administering suboptimal or even harmful cell therapies.

The typical way to prepare PRP or cBMA is via centrifugation. Similar to the way pulp is extracted from orange juice, the underlying principle is to centrifuge a volume of whole blood or bone marrow to recapture a large percentage of the desired cells from the blood or bone marrow. For a proven therapy to be beneficial, the exact amount of PRP or cBMA must be administered to the patient. The problem is, there are inherent variables that influence cell quantities all throughout the centrifugation process that inhibit the ability to deliver exact amounts. These variables are often harder to control in clinical settings than in research settings, thereby exacerbating the problem.

Several inherent variables influencing cell quantities throughout the treatment process and protocol have been identified. For example, the clinical preparation of PRP is largely pre-defined by the commercial centrifuge kit being used. However, the respective cell recovery performance efficiency of these machines varies greatly. Another example, the cell quantities/yields per mL differ across various anatomical sites. For example, the calcaneus is considered one of the poorest aspiration sites, whereby the iliac crest is considered one of the best. Depending on the specialty, a physician may be limited as to where they can anatomically aspirate or may simply be unwilling to aspirate the autologous source due to unfamiliarity of the anatomy (e.g., a podiatrist vs. a neurosurgeon). Known aspiration techniques are often labor intensive and time-consuming to execute. In addition, varying degrees of cell yields per mL may be achieved when using different syringe sizes, styles of aspiration needles, and/or duration of the aspiration.

Comparison studies have shown the wide disparity of cell recovery performance efficiency or recapture percentage among various commercially available centrifuge machines. Many centrifuge machines require human involvement following the centrifugation process, which compounds the variability because the final homogenization and preparation of the final cell concentrate is often done by unskilled technicians. Unskilled manipulation during this step greatly influences the cell concentration process and protocol, particularly the clinical setting.

Today, the number of centrifuges, preparation protocols, and processing protocols available has increased. However, an exact amount of platelets must still be utilized to achieve desired results. For example, certain targeted treatment applications might not benefit from a general collective enrichment. Rather, certain targeted treatments may benefit more from the enrichment of some factors while depleting or reducing other factors.

Similar to PRP, the clinical utility of cBMA requires exact amounts of concentrate to realize its benefits. Many organizations define cBMA as a procedure to aspirate a large volume of bone marrow which contains a collection of pluripotent cells, monocytes, platelets, VSELs, and other factors. Once aspirated, the large volume of bone marrow is then concentrated to obtain a quantitative enrichment of these collective cells over a patient’s baseline. The final concentrated enrichment, in turn, can be utilized for cell therapy treatments. The process of obtaining cBMA is similar to that of PRP, via density gradient separation. Like PRP, the current understanding and benefits of cBMA targeted cell therapy is understood. As a result, there are many centrifugation and other devices that have been created to isolate and enrich only certain factors within bone marrow for targeted therapies. Although the benefits of treatments using PRP and/or cBMA are understood, the exact concentrations and treatment protocols are still evolving.

One of the reasons that slowing the widespread adoption of PRP and cBMA treatments may be due to a lack of standardization and the inherent high degree of variability that exists for cell therapy treatments. PRP and cBMA treatments are defined as quantitative enrichments over a patient’s baseline levels. However, a “5x concentration” of a 30 mL blood draw from a patient with a baseline platelet count of 160,000/mL is significantly different than a “5x concentration” for a patient with a baseline platelet count of 340,000/mL. Many who treat or research cell therapies consider the link between obtaining the necessary enriched quantities to be solely dependent on the centrifugation device when, in fact, there are numerous variables, centrifugation being one of them, that all affect the concentrated enriched quantity. Moreover, most of the variables are present prior to centrifugation. Surprisingly, this is not largely understood or respected by the average research or treating physician. As a consequence, published studies focus too much on the centrifugation device variable and not controlling other variables.

One of the variables is aspirating the right amount of volume for the particular patient. A centrifuge cannot create cells or enhance the quantities if it does not process an appropriately large volume. Enriched cell yield, following concentration, is needed to produce a targeted therapeutic benefit. Broadly, any published, controlled research study showing a clinical benefit from using any cell therapy adheres to reported cellular target ranges to achieve positive clinical outcomes. It is well documented and known that large volumes of blood or bone marrow, differing from pre-defined commercial centrifuge kits, must be aspirated and concentrated in order to produce clinical benefits. However, it has been empirically observed over thousands of surgeries in the operating room setting, as well as documented in clinical literature, that treating physicians simply do not aspirate volumes close to those in published literature. In real-world settings, clinicians want to know how much autologous fluid they should be aspirating, however, commonly default to the pre-defined aspiration volumes of the commercial processing kit being used. Other times, physicians simply aspirate a volume they feel is appropriate or simply aspirate a constant volume for each and every patient. In any of these scenarios, treating physicians do not aspirate volumes close to achieving consistent cellular target ranges demonstrated in the correlating published literature for a particular treatment. Due to this variable, it is important for a physician to understand the pre-centrifugation aspiration volume to achieve a desired target range for a particular treatment. Understanding the starting volume would help deliver consistent, customized therapies to individual patients.

Other variables include anatomical variability from patient-to-patient and variations in aspiration techniques. Depending on the specialty, a physician could be limited as to where they can anatomically aspirate or simply unwilling to aspirate the autologous source due to unfamiliarity of the anatomy. This is a critical point because aspirating source volumes in anatomical areas that yield very few cells per mL would require to the physician to take higher volumes from the anatomical area in order to achieve appropriate cell yields following concentration. Many researching and treating physicians focus too much on the concentration device as the ultimate determining factor, yet a centrifuge cannot create cells. Controlling this particular variable prior to processing, would help standardize cell therapy treatments.

Other variables include the size of aspirating syringe or needle and the components in the processing kits. It is well demonstrated the inherent high variability with different volumes of syringes, aspiration trocars, and needles with respects of the amount of cells or platelets/mL. For example, aspirating 10% of a 10 cc syringe (1 cc) may yield the highest amount of progenitor cells when comparing different percentages of different sized syringes. Due to the negative pressure physics of a 10 cc syringe (pressure = force/area), the counterintuitive physics demonstrated that a 50 mL syringe yielded lower progenitor cells at every aspirating percentage. If a treating physician were to adhere to this in a real-world setting, it would take 30-45 minutes to aspirate a minimum concentrating volume. Despite the variability, it is simply unrealistic for any physician to take that much time. Furthermore, many commercial processing kits do not contain 1 cc syringes for aspirating. It is important for a physician to control these variables regardless of the syringe size, needle size, or trocar style.

Other variables include overall patient biology and centrifugation technology. It is well understood that there is significant variation in the capture efficiency of commercial centrifugation systems and biological variation in the source population due to differences in patient age, health and nutritional status. Over the years, there have been technological advancements in centrifugation systems resulting in improved viability, recovery performance / recapture percentage, and number of cells delivered over baseline to name a few. However, despite these performance advancements, many commercially available machines require tremendous manual involvement during concentration and extraction phases which equates to a high degree of variability. There are very few concentration systems that are completely automated during processing and post-concentration extraction. Any human involvement during processing or extraction of the cells introduces variability even for a skilled person. Cell concentration machines, in theory, are designed to have a reproducible linear performance. Concentration systems that require human manual involvement during the process, particularly the critical phase of concentration and extraction, creates tremendous translation gaps in the effort to standardize cell therapy treatments. The majority of published cell therapy studies do not take this into account even though a target cell enrichment quantity is needed to produce desired therapeutic benefits.

Despite the known benefits of cell therapies, there exists a lack of standardization of treatments and translation from the research settings into the real world clinical settings. Research in this area is updated daily with dozens of cell therapy articles creating an enormously vast database of published clinical literature that is virtually impossible for a physician to stay current of the best treatment options. The tools and resources used in controlled research settings are costly, not feasible, and not available for the everyday treating physician to reproduce published results. Furthermore, the literature is divided into concentration machine performance studies, clinical outcomes studies, different autologous sources utilized to treat the same conditions, different schools of thought, and much more. The lack of standardization and high degree of variability has created a huge information gap between the research setting and real-world clinical setting.

### SUMMARY OF THE INVENTION

This invention relates in general to the field of medical treatments. In various embodiments, the system may calculates a pre-draw aspiration volume needed for centrifugation to achieve a concentrated target threshold dose for a particular cell therapy, allowing physicians to customize, personalize, and translate consistent cell therapies to patients according to its proven, scientific dosing threshold. In various embodiments, the system may enable physicians to translate and reproduce proven cell therapies in a clinical setting. In various embodiments, the system may calculate a target aspirate volume following a simple cell analysis. In the event anatomical or aspiration technique influences the overall target population in the aspirated target volume, then another sample analysis may be run and to calculate if additional volume is needed. In various embodiments, the system may calculate a pre-draw aspiration volume based on this preliminary cell analysis. In various embodiments, the system may facilitate processing the correct source volume needed to achieve a targeted cell therapy treatment for each individual patient. In various embodiments, anatomical and technique variables may be controlled inherently by insuring the appropriate volume has been aspirated and the overall cell population is present prior to centrifugation. In the event syringe size, needle size, or trocar style influence the cell population, then another sample analysis may be run to determine if additional autologous fluid is needed. In various embodiments, the system can determine a desired therapeutic cell concentration target needed by accessing a published literature database or real-time user input. In various embodiments, once the cell concentration target number is known, a cell analysis may be sampled and the centrifugation device may be inputted, then the system may calculate the target aspirated volume needed from the individual patient. Following concentration, a quick cell analysis may also determine if manual involvement or machine performance influenced the concentrated target quantity needed. Should this occur, the system may then reverse calculate the volume needed to be concentrated to obtain the desired target cell concentration number or calculate a dilution volume to obtain the desired therapy quantity target. In the reverse calculation, the system may factor in that this will be mixed and homogenized with the first concentration in order to insure the final concentrate for treatment contains the appropriate quantity of target cells/mL needed to produce therapeutic benefit. In the dilution calculation, the system may factor in that this will be mixed and homogenized with supernatant, or other fluid at the discretion of the user, to insure the final concentrate contains the appropriate quantity of target cells/mL.

In various embodiments, systems and methods are provided for quick and efficient cell analysis and calculations to reduce and/or eliminate the potential variables prior to centrifugation to insure consistency from patient to patient for targeted cell therapy treatments. In various embodiments, an appropriate volume to be aspirated may be recommended to a physician by factoring in various information such as, the desired treatment with associated known target range, the concentration machine, and the amount of treatment volume the physician is needing. In various embodiments, the system may include a point-of-care device designed to calculate the volume of autologous source needed in order to deliver the desired target range of cells/mL. In some embodiments, the system may guide a physician down a series of workflow algorithm prompts in accordance with published literature, providing the physician with selections and information in the point of care setting in order to facilitate informed medical treatment decisions for each individual patient.

In accordance with one aspect of the present invention, a system and method for validating personalized cell therapy treatments is disclosed. In one embodiment, the diagnostic system and method may provide verification of the cell treatment algorithmically, while simultaneously identifying, through published clinical literature, the appropriate cell type and quantity of cells shown to be effective. In various embodiments, the system may be agnostic to the specific concentration systems being used. Furthermore, the open structure design may allow embodiments of the tool to be used compatibly with any cell counter. Following a small aliquot autologous sample, a cell analysis is performed to understand the baseline cells/mL for individual subjects. Next, the system uses a workflow prompt of selections to understand specialty, intended treatment, autologous source being used, concentration machine being used, and treatment volume needed. The workflow prompt selections are designed to inform treating physicians of options from published literature and to determine the necessary target cell/mL volume needed for the desired treatment in order to calculate the volume of autologous source needed to reproduce proven results in the research settings to the clinical treatment settings. The diagnostic tool may contain embedded calculations to determine the appropriate source volume needed for concentrating in any centrifuge or mechanical cell separating device. Following concentration, the tool may quantitatively verify the intended cell phenotype target has been concentrated, calculate any dilution needed, and calculate any additional volume needed to be concentrated prior to the treating physician administering cell therapy treatment. In all, the system provides the real-world physician a tool that facilitates quality control, consistency, and reproducibility of treatment results from the research setting.

In various embodiments, a diagnostic computing device, such as a computer or tablet PC, may be used to provide an algorithmic verification of the intended cell treatment through a prescribed work-flow to guide a physician through a series of prompts about the procedure and intended treatment they wish to execute. The work-flow may be structured such that it may limit the prompts to those correlating to predetermined published literature or other factors the physician or service provider may choose or define. In some embodiments, the published literature utilized to guide the work-flow prompts may be derived from software embedded into the hard drive of the diagnostic device with updates of literature coming from external devices or may be accessed from a remote source in real time, such as through the Internet, the cloud, a local area network, an external server, via a Bluetooth connection or external router. In some embodiments, the diagnostic computing device may be configured to interface with proprietary centrifuges, various commercially available centrifuges, and/or various cell counter devices. The diagnostic computing device may be built into such devices or interface with them via a hardwire connection and/or via Bluetooth or other wireless interface, or using a USB or other capabilities.

Various embodiments may include a diagnostic computing device, wherein the device is capable of calculating the appropriate source volume needed for aspiration prior to concentration. The diagnostic computing device may be compatible with any commercially available centrifuge device and associated kit parts. The diagnostic computing device may ensure the source volume needed correlates to the appropriate targeted cell/mL needed for the intended treatment. The diagnostic computing device may ensure cell yields for intended treatment are consistent with published literature, user defined parameters, or crowd-sourced information. The diagnostic computing device may allow cell yields and intended treatment to be determined by the specific source prompted by the physician. The diagnostic computing device may, following concentration, validate the appropriate cell quantity and phenotype for the intended treatment were isolated for cell treatment. The diagnostic computing device may quantify any additional source volume needed to be concentrated in order for the cell treatment to be consistent with quantities and phenotype concentrations shown in published literature.

Various embodiments may include a diagnostic computing device that is compatible with EMR systems in order to be used for clinical follow up and research purposes, and/or wherein the device is compatible with registry-based software and applications to be used for follow up and research purposes. In some embodiments, the diagnostic computing device and/or information collected using such device may be transferrable to clinical or research purposes when administering cell therapy to animals.

The above summary of the invention is not intended to represent each embodiment or every aspect of the present invention. Particular embodiments may include one, some, or none of the listed advantages.

### BRIEF DESCRIPTION OF THE DRAWINGS

A more complete understanding of the method and apparatus of the present invention may be obtained by reference to the following Detailed Description when taken in conjunction with the accompanying Drawings wherein:

FIG. 1 is a diagram of a computer network according to one embodiment of the present invention;

FIG. 2 is a diagram providing additional details of an embodiment of a computer network;

FIG. 3 is a flowchart of a method according to an embodiment;

FIG. 4 is a flowchart of a method according to an embodiment;

FIG. 5 is a screenshot of a user interface according to an embodiment; and

FIG. 6 is a screenshot of a user interface according to an embodiment.

### DETAILED DESCRIPTION

The present invention is directed towards systems and methods for increasing successful outcomes in cell therapy treatments. Currently, point-of-care cell therapy lacks sufficient standardization. In various embodiments, systems and methods are provided for making the latest treatment protocols available to doctors, nurses, and other technicians at the point-of-care. When conducting a cell therapy treatment study in a controlled setting, several safety measures may be in place to ensure accuracy that may not be in place in a real-world setting. In both the research and real-world settings, cell therapy treatments generally include a physician aspirating a determined large volume of autologous fluid from a subject, concentrating this fluid via centrifugation to obtain a final small volume of concentrate, and then injecting this small volume concentrate to a target site. In further embodiments, systems and methods are provided for increasing the accuracy of a target concentration for various treatment protocols by analyzing various factors present in the real-world setting. In various embodiments, systems and methods are provided that may increase the reliability within a cell therapy treatment by addressing variables such as, for example, a physician’s aspiration technique, patient baseline, patient health status, pipetting technique, sizes of the syringes used during aspiration, machine centrifugation performance, cell analysis, time between aspiration, autologous source volume, and other real-world factors.

Clinical outcomes are more likely to succeed when a target cell/mL is achieved prior to centrifugation. In order for one to be sure enough total cells are present to centrifuge, despite all of the other variables, one must aspirate the appropriate volume from the patient to reach a desired target cell/mL in the final volume of concentrate. Physicians often determine their aspirating volume by the centrifuge kit volume limitations, habitually aspirate the same volumes for each patient, or stop aspirating when they feel they have enough. In various embodiments, systems and methods are provided that may help to ensure the appropriate amount of target cells/mL has been achieved in the final small volume of concentrate. By way of example, different treatment protocols may require different target cell/mL to achieve therapeutic benefits. Oftentimes, blood is aspirated and then passed off for centrifugation to be used later. Various embodiments may include prompting a technician with the specific volume needing to be aspirated, which would be the appropriate volume to achieve the concentrated treatment target cell/mL called for in the published literature.

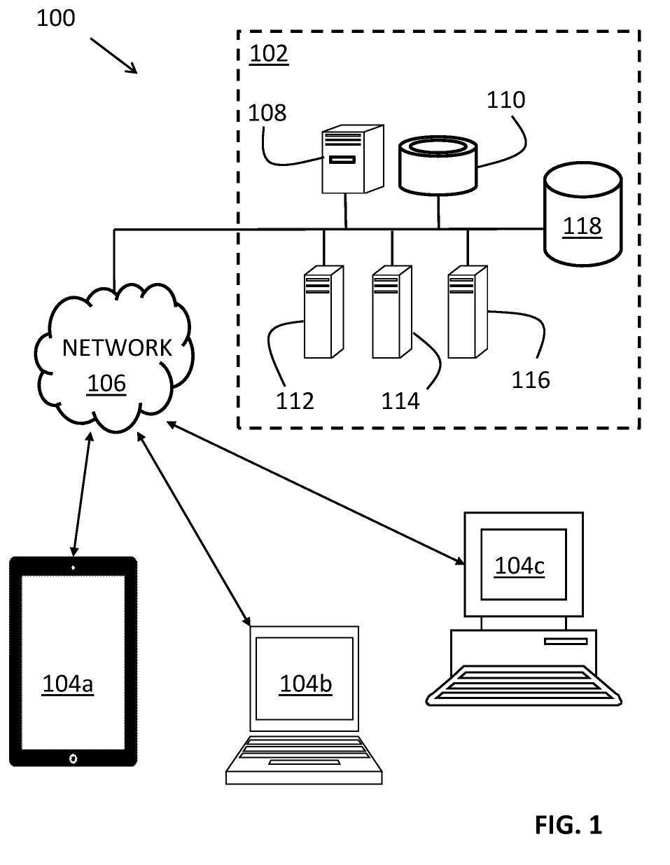

Referring now to FIG. 1, a schematic diagram according to an example embodiment is shown illustrating a computer network system 100 having a client-server architecture. The computer network system 100 includes a treatment protocol system 102 and one or more client computers 104a-c, communicatively coupled via a network 106. In an embodiment, the treatment protocol system 102 may include a cell counter 108 (or similar device, such as a platelet counter or a hemacytometer), a centrifuge 110, a web server 112, a plurality of other servers 114-116, such as, for example, web servers, application servers, messaging servers, database management servers, and file servers, and a storage device 118. The treatment protocol system 102 may be implemented as a distributed system. For example, one or more elements of the treatment protocol system 102 may be located across a wide-area network from other elements of the treatment protocol system 102. As another example, a server may represent a group of two or more servers, cooperating with each other, in providing a pooled, distributed, or redundant computing model. Moreover, those skilled in the art will appreciate that one or more embodiments may be practiced with any number of computer system configurations including, but not limited to, where program modules may be located in local and/or remote memory storage devices.

The client computers 104a-c may include electronic devices in various forms, such as a mobile communication device 104a (e.g., a PDA, a smart phone, a tablet computer, etc.), a laptop computer 104b, a desktop computer 104c, or other devices capable of network communication and visual presentation. In addition, the client computers 104a-c may include a thin-client, a thick-client, a fat client, a hybrid client, or other client model typically found in a client-server architecture. Although not shown, one or more of the client computers may be the user interface of a cell counter, a centrifuge, or other medical device, or may be coupled directly thereto. The network 106 may include local-area networks (LAN), wide-area networks (WAN), wireless networks, the Internet, or other combinations or permutations of network protocols and network types. The web server 112, either alone or in conjunction with one or more other computers in the treatment protocol system 102, may provide a user interface.

In an embodiment, client computer 104c may be an administrative user. During operation, the admin user may access the treatment protocol system 102 to upload content to be published to users. The treatment protocol system 102 may then convert the uploaded content from a first format into a workflow format. The admin user may then request that the workflow published to a virtual library to allow authorized user access to the workflow.

In an embodiment, the client computer 104a may include a client program (e.g., a workflow program or module) to interface with the treatment protocol system 102. The client program may include commercial software, custom software, open source software, freeware, shareware, or other types of software packages. The client program may interact with a server program hosted on a server of the treatment protocol system 102. During operation, a user at a client computer 104a can access the treatment protocol system 102 to view a workflow for a specific treatment protocol. The treatment protocol may be downloaded to the client computer 104a or may remain in the treatment protocol system 102 and viewed remotely.

The user device 104a may display a workflow prompt process in order to determine if the clinician is collecting cells for research purposes or non-research purposes. If used for non-research purposes, then the workflow may prompt selections that are consistent with published literature stored in storage device 118. The user device 104a may interact with a storage device 118, which may be a local server, remote server, cloud based server, or other device in order to access the latest published literature. Cell therapies advance at a fast pace making it virtually impossible for treating physicians to be current on the most up-to-date literature. Therefore, the workflow prompt may be configured to only provide selections to the physician that are consistent with published literature. This insures that physicians have point-of-care information to make informed treatment decisions. In some embodiments, for example in research settings, the published literature may not be needed, but the treatment protocol system 102 may still be used to calculate the autologous volume needed.

While the system 100 shown in FIG. 1 employs a client-server architecture, the present invention is of course not limited to such an architecture, and could equally well find application in a distributed, or peer-to-peer, architecture system, for example. Additionally, while various computers and storage devices are illustrated as separate components in FIG. 1, it is understood that functionality of these components may be merged, distributed, or otherwise organized into different configurations, depending on the implementations due to design preferences, cost restrictions, geographical limitations, or other business, technical, or practical considerations.

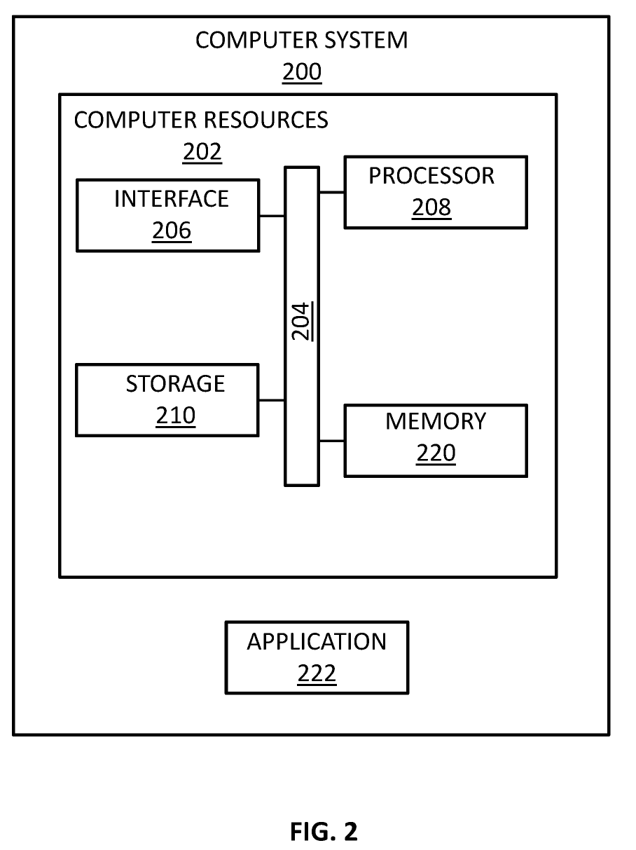

Referring now to FIG. 2, a diagram of a computer system 200 for implementing and connecting the user devices 104a-c to the treatment protocol system 102, including, for example, cell counter 108, centrifuge 110, servers 112-116, storage device 118, and/or other computers and devices is provided. The components of the computer system 200 may comprise any suitable physical form, configuration, number, type and/or layout. As an example, and not by way of limitation, the computer system 200 may comprise an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a wearable or body-borne computer, a server, or a combination of two or more of these. Where appropriate, the computer system 200 may include one or more computer systems; be unitary or distributed; span multiple locations; span multiple machines; or reside in a cloud, which may include one or more cloud components in one or more networks.

In the depicted embodiment, the computer system 200 includes a processor 208, memory 220, storage 210, an interface 206, and bus 204. Although a particular computer system is depicted having a particular number of components in a particular arrangement, this disclosure contemplates any suitable computer system having any number of suitable components in any arrangement.

Processor 208 may be a microprocessor, controller, or any other suitable computing device, resource, or combination of hardware, software and/or encoded logic operable to execute, either alone or in conjunction with other components (e.g., memory 220), the application 222. Such functionality may include providing various features discussed herein. In particular embodiments, processor 208 may include hardware for executing instructions, such as those making up the application 222. As an example and not by way of limitation, to execute instructions, processor 208 may retrieve (or fetch) instructions from an internal register, an internal cache, memory 220, or storage 210; decode and execute them; and then write one or more results to an internal register, an internal cache, memory 220, or storage 210.

In particular embodiments, processor 208 may include one or more internal caches for data, instructions, or addresses. This disclosure contemplates processor 208 including any suitable number of any suitable internal caches, where appropriate. As an example and not by way of limitation, processor 208 may include one or more instruction caches, one or more data caches, and one or more translation lookaside buffers (TLBs). Instructions in the instruction caches may be copies of instructions in memory 220 or storage 210 and the instruction caches may speed up retrieval of those instructions by processor 208. Data in the data caches may be copies of data in memory 220 or storage 210 for instructions executing at processor 208 to operate on; the results of previous instructions executed at processor 208 for access by subsequent instructions executing at processor 208, or for writing to memory 220, or storage 210; or other suitable data. The data caches may speed up read or write operations by processor 208. The TLBs may speed up virtual-address translations for processor 208. In particular embodiments, processor 208 may include one or more internal registers for data, instructions, or addresses. Depending on the embodiment, processor 208 may include any suitable number of any suitable internal registers, where appropriate. Where appropriate, processor 208 may include one or more arithmetic logic units (ALUs); be a multi-core processor; include one or more processors 208; or any other suitable processor.

Memory 220 may be any form of volatile or non-volatile memory including, without limitation, magnetic media, optical media, random access memory (RAM), read-only memory (ROM), flash memory, removable media, or any other suitable local or remote memory component or components. In particular embodiments, memory 220 may include random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM, or any other suitable type of RAM or memory. Memory 220 may include one or more memories 220, where appropriate. Memory 220 may store any suitable data or information utilized by the computer system 200, including software embedded in a computer readable medium, and/or encoded logic incorporated in hardware or otherwise stored (e.g., firmware). In particular embodiments, memory 220 may include main memory for storing instructions for processor 208 to execute or data for processor 208 to operate on. In particular embodiments, one or more memory management units (MMUs) may reside between processor 208 and memory 220 and facilitate accesses to memory 220 requested by processor 208.

As an example and not by way of limitation, the computer system 200 may load instructions from storage 210 or another source (such as, for example, another computer system) to memory 220. Processor 208 may then load the instructions from memory 220 to an internal register or internal cache. To execute the instructions, processor 208 may retrieve the instructions from the internal register or internal cache and decode them. During or after execution of the instructions, processor 208 may write one or more results (which may be intermediate or final results) to the internal register or internal cache. Processor 208 may then write one or more of those results to memory 220. In particular embodiments, processor 208 may execute only instructions in one or more internal registers or internal caches or in memory 220 (as opposed to storage 210 or elsewhere) and may operate only on data in one or more internal registers or internal caches or in memory 220 (as opposed to storage 210 or elsewhere).

In particular embodiments, storage 210 may include mass storage for data or instructions. As an example and not by way of limitation, storage 210 may include a hard disk drive (HDD), a floppy disk drive, flash memory, an optical disc, a magneto-optical disc, magnetic tape, or a Universal Serial Bus (USB) drive or a combination of two or more of these. Storage 210 may include removable or non-removable (or fixed) media, where appropriate. Storage 210 may be internal or external to the computer system 200, where appropriate. In particular embodiments, storage 210 may be non-volatile, solid-state memory. In particular embodiments, storage 210 may include read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. Storage 210 may take any suitable physical form and may comprise any suitable number or type of storage. Storage 210 may include one or more storage control units facilitating communication between processor 208 and storage 210, where appropriate.

In particular embodiments, interface 206 may include hardware, encoded software, or both providing one or more interfaces for communication (such as, for example, packet-based communication) among any networks, any network devices, and/or any other computer systems. As an example and not by way of limitation, communication interface 206 may include a network interface controller (NIC) or network adapter for communicating with an Ethernet or other wire-based network and/or a wireless NIC (WNIC) or wireless adapter for communicating with a wireless network.

Depending on the embodiment, interface 206 may be any type of interface suitable for any type of network for which computer system 200 is used. As an example and not by way of limitation, computer system 200 can include (or communicate with) an ad-hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 200 can include (or communicate with) a wireless PAN (WPAN) (such as, for example, a BLUETOOTH WPAN), a WI-FI network, a WI-MAX network, an LTE network, an LTE-A network, a cellular telephone network (such as, for example, a Global System for Mobile Communications (GSM) network), or any other suitable wireless network or a combination of two or more of these. The computer system 200 may include any suitable interface 206 for any one or more of these networks, where appropriate.

In some embodiments, interface 206 may include one or more interfaces for one or more I/O devices. One or more of these I/O devices may enable communication between a person and the computer system 200. As an example and not by way of limitation, an I/O device may include a keyboard, keypad, microphone, monitor, mouse, printer, scanner, speaker, still camera, stylus, tablet, touchscreen, trackball, video camera, another suitable I/O device or a combination of two or more of these. An I/O device may include one or more sensors. Particular embodiments may include any suitable type and/or number of I/O devices and any suitable type and/or number of interfaces 206 for them. Where appropriate, interface 206 may include one or more drivers enabling processor 208 to drive one or more of these I/O devices. Interface 206 may include one or more interfaces 206, where appropriate.

Bus 204 may include any combination of hardware, software embedded in a computer readable medium, and/or encoded logic incorporated in hardware or otherwise stored (e.g., firmware) to couple components of the computer system 200 to each other. As an example and not by way of limitation, bus 204 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCI-X) bus, a serial advanced technology attachment (SATA) bus, a Video Electronics Standards Association local (VLB) bus, or any other suitable bus or a combination of two or more of these. Bus 204 may include any number, type, and/or configuration of buses 204, where appropriate. In particular embodiments, one or more buses 204 (which may each include an address bus and a data bus) may couple processor 208 to memory 220. Bus 204 may include one or more memory buses.

Herein, reference to a computer-readable storage medium encompasses one or more tangible computer-readable storage media possessing structures. As an example and not by way of limitation, a computer-readable storage medium may include a semiconductor-based or other integrated circuit (IC) (such, as for example, a field-programmable gate array (FPGA) or an application-specific IC (ASIC)), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, a flash memory card, a flash memory drive, or any other suitable tangible computer-readable storage medium or a combination of two or more of these, where appropriate.

Particular embodiments may include one or more computer-readable storage media implementing any suitable storage. In particular embodiments, a computer-readable storage medium implements one or more portions of processor 208 (such as, for example, one or more internal registers or caches), one or more portions of memory 220, one or more portions of storage 210, or a combination of these, where appropriate. In particular embodiments, a computer-readable storage medium implements RAM or ROM. In particular embodiments, a computer-readable storage medium implements volatile or persistent memory. In particular embodiments, one or more computer-readable storage media embody encoded software.

Herein, reference to encoded software may encompass one or more applications, bytecode, one or more computer programs, one or more executables, one or more instructions, logic, machine code, one or more scripts, or source code, and vice versa, where appropriate, that have been stored or encoded in a computer-readable storage medium. In particular embodiments, encoded software includes one or more APIs stored or encoded in a computer-readable storage medium. Particular embodiments may use any suitable encoded software written or otherwise expressed in any suitable programming language or combination of programming languages stored or encoded in any suitable type or number of computer-readable storage media. In particular embodiments, encoded software may be expressed as source code or object code. In particular embodiments, encoded software is expressed in a higher-level programming language, such as, for example, C, Perl, or a suitable extension thereof. In particular embodiments, encoded software is expressed in a lower-level programming language, such as assembly language (or machine code). In particular embodiments, encoded software is expressed in JAVA. In particular embodiments, encoded software is expressed in Hyper Text Markup Language (HTML), Extensible Markup Language (XML), or other suitable markup language.

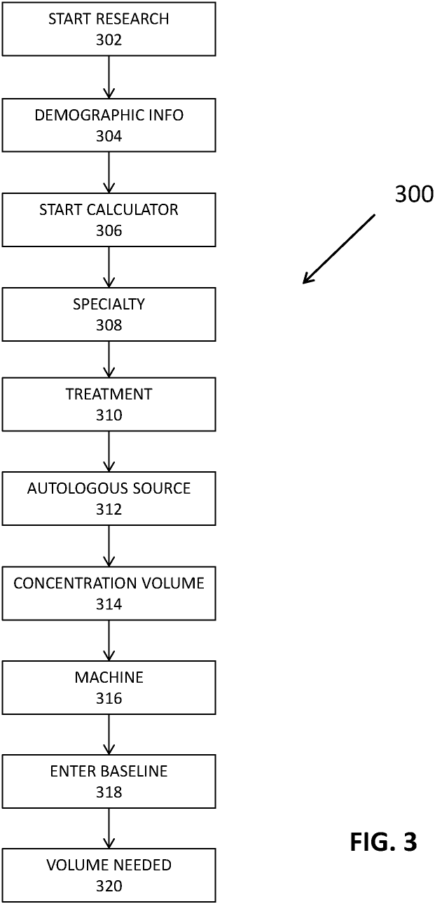

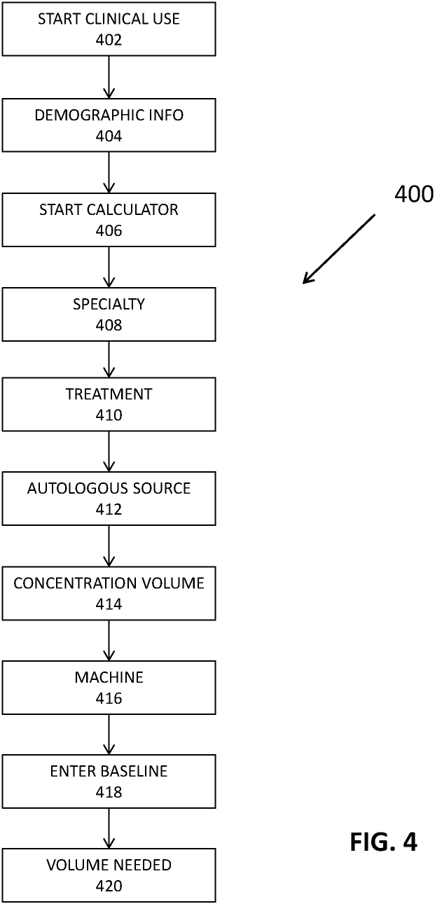

Referring now to FIGS. 3 and 4, flowcharts are provided of an embodiment of a method 300 of providing a treatment protocol using a user device. At steps 302 and 402, a user is prompted to select whether the use will be for research purposes or for non-research purposes. In various embodiments, at steps 304 and 404, patient information and/or de-identified demographics of the intended subject could be inputted or selected from a drop down menu, such as, for example, whether the subject is human or animal, the sex of the subject, age, name, initials, or other indicia, treating physician, facility, location, and other relevant information. Such information may be useful for tracking, data and research collection purposes. These selections may be inputted and displayed via the user device.

At steps 306 and 406, the user begins the calculation workflow. At steps 308 and 408, the user device receives input from the physician regarding specialty. If being used for non-research purposes, then the user device may be programmed to prompt specialty selections that are within the published literature showing cell therapy human outcomes within the specified specialty. If the specialty is not reported in published literature or not published with human outcomes, then, at step 308, the physician may be prompted to add the specialty before proceeding through the workflow prompt. When a new specialty is added, the physician may be notified by the user device that it will no longer be accessing the cloud based and/or embedded published outcomes. The device may still proceed through the workflow prompts and calculate the needed autologous volume, however, the physician may be prompted that the volume calculated is intended for an experimental specialty use not reported in the published literature. In various embodiments, the physician may be required to acknowledge this before proceeding to the next workflow prompt. If the user device is being used for research purposes, then the physician may input a designated specialty. These selections are displayed and received via the user device.

At steps 310 and 410, the device receives input from the physician regarding an intended treatment. If being used for non-research purposes, then the device may prompt treatment selections based at least in part on the specialty selection that are within published literature showing cell therapy human outcomes. If the intended treatment is not reported in published literature or not published with human outcomes, then the physician will be prompted to add the treatment before proceeding through the workflow prompt. If a treatment is added, then the physician may be notified by the machine that it will no longer be accessing the cloud based and/or embedded published outcomes. The device may still proceed through the workflow prompts and calculate the needed autologous volume, however, the physician may be prompted that the volume calculated is intended for an experimental treatment use not reported in the published literature. The physician may need to acknowledge this before proceeding to the next workflow prompt. If the device is being used for research purposes, then the physician may input the designated specialty.

At steps 312 and 412, the device receives input from the physician regarding an intended autologous source. If being used for non-research purposes, the device may prompt autologous source selections, determined from the specialty selection, that are within published literature showing cell therapy human outcomes. Additionally, underneath the autologous source selection may be a strength number. In such embodiments, the strength number is based at least in part on the amount of literature published for the autologous source. Due to the rapid advancement of published literature and research using different autologous sources for the same treatment, it is difficult for physicians to stay current on the best treatment options. The strength number is meant to show numerically how one autologous source compares to another autologous source within the published literature. If clarification is needed, the physician can select the strength number, whereby, a list of the studies and abstracts are displayed showing the clinical evidence behind the strength number. If further clarification is needed, the physician can select a study title to read the complete study. This information is valuable in the clinical setting by allowing the physician to be better informed when making cell therapy treatment decisions. If the intended source is not reported in published literature or not published with human outcomes, then the physician may be prompted to add the source for the selected treatment before proceeding through the workflow prompt. If add autologous source is selected and inputted, then the physician may be notified by the user device that it may no longer be accessing the cloud based and/or embedded published outcomes. The device may still proceed through the workflow prompts and calculate the needed autologous volume, however, the physician may be prompted that the volume calculated is intended for an experimental treatment use not reported in the published literature. The physician may need to acknowledge this before proceeding to the next workflow prompt.

At steps 314 and 414, the device receives input from the physician regarding concentration volume needed. If being used for non-research purposes, the device may only prompt a default numerical concentration milliliter volume, determined from the treatment selection. The defaulted volume may be based at least in part on the relevant published literature. The treatment targeted cell range per mL is displayed for the physician to view and confirm. In various embodiments, different treatments may have different numerical target ranges that have shown to be clinically effective in the published literature. The concentrated volume needed is a critical value that influences the necessary numerical target cell/mL value shown in published literature. The physician may have the option to increase or decrease the concentration volume by selecting plus (+) and minus (-) signs. The value for the starting volume needed to achieve the concentration volume needed for treatment is calculated based at least in part on the different inputs received during the workflow. Any number of calculations can be used to determine starting volume needed to maintain target cell/mL necessary for the intended treatment. Changing various inputted values will affect the resulting calculations.

At steps 316 and 416, the device receives input from the physician regarding the concentration machine being used. If being used for non-research purposes, the device may prompt concentration machines with known performance value criteria. The performance criteria is another example of a value that may influence the starting volume calculation. This is due to the known studied performance variabilities of commercial cell concentration devices. The physician may also have the option to add a machine. When adding a machine, a weighted performance average may be calculated to determine the starting volume calculation. In this scenario, the device may notify the physician that a weighted average is being used to determine the final calculations and not a known performance for the added machine. The physician may be prompted to acknowledge using a machine performance not known within the published literature before proceeding.

At steps 318 and 418, the user device receives baseline cell numbers that will be used for calculations. The baseline cell number can be taken by any commercially available cell counter, platelet counter, hemacytometer, or like device. The user device may receive the baseline numbers via manual input or via wired or wireless connection to the counter and/or other backend system to determine which calculation should be performed: for platelets, RBCs, HSCs, WBCs, exosomes, adipose pre-cursor cells, or MSCs. The user device can also be connected, either wired or wirelessly, to capable cell counting devices in order to transfer baseline data instead of manual input. These selections are displayed and received via the user device. In the embodiment shown, the baseline cell number is inputted after various other information has been entered. Information provided by the system based at least in part on user inputs may assist the user in determining the source material to be used in the treatment. Because the baseline may vary depending on the source material, in various embodiments, although not required, it may be preferable for a user to input other information (e.g., specialty, treatment, and/or autologous source) before determining and/or inputting the baseline cell number.

At steps 320 and 420, the device displays the starting volume amount of autologous source volume needed to be aspirated from the subject. This final calculation is determined based at least in part on the previous inputs from the clinician. This starting volume is the final calculated volume needed from the individual subject, to be concentrated, in order to concentrate a final treatment volume containing the target cell/mL range needed, according to published literature, to achieve the treatment results shown in published literature.

In some embodiments, the device may also determine dilution and/or hyperconcentration calculations. Dilution and/or hyperconcentraton calculations may be an important value to know following machine concentration. By way of example, in various embodiments of the system, the starting volume was calculated to ensure an appropriate target cell/mL treatment yield is achieved. Following machine concentration, a physician can test a small aliquot sample of the concentrate. If the target cell/mL volume exceeds the intended target, the excess plasma or other fraction of the separation can be added to dilute the concentrate in order to achieve the intended target cell/mL. The treatment system may calculate exactly how much dilution should be added. The opposite would occur if too little target cells/mL were achieved. If the target cell/mL is less than the intended target treatment need, then hyperconcentration would need to occur. In this case, a cell analysis of the concentrate would be used by the system to make the hyperconcentration calculations. The system would calculate the amount of excess plasma or separated fraction to be removed from the concentrate. This would yield a total volume less than the desired treatment volume, but would be at the desired treatment target cell/mL. If the literature indicated that the concentration was more important that total injection volume, hyperconcentration may be desirable. If the literature indicated that the total injection volume was more important that the concentration, then the additional hyperconcentration may not be needed. Any number of weighted algorithms and calculations could be used to determine the final volume needed or other values within the equation.

Various embodiments of the diagnostic system of the present invention provide an algorithmic set of prompts for the physician to enter. Each prompt may only display choices for the physician to choose that are in published literature. Each prompt may work successively with the previous prompts while simultaneously providing a funnel-like approach for the physician to verify that the treatment is in accordance with published literature. At the home screen, for example, the physician may input their respective specialty, e.g., orthopedics. Next, the display screen may prompt the physician to select from the treatment choices that are published in the literature. These may be treatment choices that are published in literature. The individual choices may be compiled from scientific articles and/or may be compiled from previous clinical data. The treatment choices may exclude studies in which the study author(s) reported poor outcomes or recommended against a specific cell therapy treatment. The treating physician may then select the appropriate treatment prompt, e.g., orthopedics➔arthroplasty. Once the specific treatment is selected, the next choice may be the autologous source. If a particular treatment has been studied using different autologous sources (e.g., bone marrow concentrate, PRP-LR or PRP-LP), then a “strength number” may be displayed below each autologous source selection icon. This strength number may represent the number, reputation, individual preference, respectability, reliability, or other indicator of strength of the published articles showing positive clinical outcomes for each autologous source for the specific treatment.

In various embodiments, this strength number may have one or more benefits. First, it may provide important information real time to verify the appropriate current treatment algorithm prior to treatment. The strength number may serve as a quick identifier to a physician of the updated literature and to validate one’s decision tree should the strength of evidence change. For example, in response to the following selections: specialty➔orthopedics; treatment➔Soft Tissue Rotator Cuff; the diagnostic device could provide the following information to the physician: Source➔Bone Marrow Concentrate [2]; Platelet Rich Plasma-LR [13]; Platelet Rich Plasma-LP [1]. PRP-LR would likely be the one selected because the strength number indicates the amount of support for this selection from the published literature. At a later date, the strength number may have changed to: Source➔Bone Marrow Concentrate [9]; Platelet Rich Plasma-LR [13]; Platelet Rich Plasma-LP [3]. The system may provide an indication of the trend or the physician may simply take notice of the increase in literature for bone marrow concentrate.

In various embodiments, the strength number can be selected by the physician in order to review the individual abstracts that comprise the individual strength number. This becomes extremely valuable real time information to a physician who is unaware of the rapid publishing of literature. The ability to review real time published information allows for a physician to administer treatments with the most up-to-date information.

Once the physician has selected the autologous source they wish to administer, the system then prompts the physician to select the concentration machine the physician will be using. In various embodiments, the system uses the individual machine’s performance variability in calculating the starting volume needing to be drawn from the patient.

In one aspect, the proposed diagnostic system calculates the minimal amount of autologous blood needed for a specific treatment for a specific patient. Currently, there are no devices for PRP cell therapy that provide up to date therapy verification and methodology translation. Various embodiments of the proposed diagnostic systems and methods can accomplish both of these. Regarding translation methodology, PRP standardization needs to report absolute quantity of platelets, injected volume, and actual platelet concentrations. A normal platelet count may range from 150,000 to 350,000 platelets per mL. Many treatment protocols call for a concentration of platelets between 1-1.5 billion platelets per mL. Platelet counts greater than 2 billion platelets per mL may be inhibitory to tenocyte behavior. Knowing the optimal concentration range is not helpful unless the amount of injected PRP volume, the concentration machine used, the starting volumes used to concentrate, and/or the absolute value of platelets is also known. Aspirating the same amount of blood in each patient is not a scientifically sound way to reach the optimal concentration range. For example, if one patient has a baseline of 150,000 platelets per mL and another has a baseline of 350,000 platelets per mL, then aspirating the same volume creates a significant variable. Furthermore, different PRP treatments require different injection amounts. For example, critical limb ischemia applications call for 20 mL PRP injections, whereas chondral lesions call for 6 mL injections. Without the literature reporting injection volumes, knowing the optimal concentration is virtually meaningless.

Today, with technology advancements, there are a number of cell counting devices available that vary in size, portability, and expense. With these advancements, this provides one of the tools that would be needed in order to translate methodologies. However, simply being able to count platelets does not provide the critical information a treating physician would need to know. Various embodiments of the present systems and methods incorporate cell counters to increase the overall treatment process. For example, prior to treatment, the proposed system may guide a physician through a set of treatment verification prompts in order to understand the treatment, specialty, and purpose of the treatment. These prompts help the physician navigate the various protocols available and also help verify to the physician they are using cell therapy treatments in accordance with up-to-date publically available literature. Next, the system needs to understand the volume injection needed to administer the treatment. At this screen, the physician can enter a desired injection volume or an injection volume is displayed on the screen according to what the literature has established for the entered treatment. Once the injected volume is entered, at this point, the proposed diagnostic system is now ready for the baseline blood sample. The diagnostic system uses the individual’s platelet count to calculate the minimal amount of source volume needed for aspiration in order to achieve the target platelet count per milliliter of injected volume for the treatment. In various embodiments, the diagnostic system may calculate the source volume based on a patient’s history, history of other patients of the same doctor or same demographic, published literature, and/or a physician’s own preferences. The diagnostic system may include connection capabilities to accommodate the majority of commercial cell counters. If the counting device does not have connectivity, the absolute platelet value can simply be manually entered into the diagnostic device. Now, the diagnostic system calculates the minimal source volume of blood needed for concentration.

This calculation may also take into account machine performance. Following concentration, another sample analysis can be taken for the diagnostic system to determine the absolute number of platelets concentrated; the number of platelets over a patient’s baseline; the number of platelets/mL of injection; and/or confirmation if the platelets per mL target has been achieved. If the target concentration has not been achieved for the desired injection volume of the given procedure, the diagnostic system may also calculate and display the additional volume of blood needed to achieve the target cell concentration. In various embodiments, the diagnostic system may automatically adjust the anticipated concentration from a particular centrifuge or technician based on historical concentrations achieved from that centrifuge, that model of centrifuge, and/or that technician. In various embodiments, the diagnostic system may provide a physician with the ability to track the progress of a patient over time. For example, a physician may be prompted to rate the results achieved by a particular treatment protocol. The physician may enter a number or other indicia or photograph of the progress. The physician may be provided with a summary of results in order to assess which protocols are successful and which may need to be adjusted.

Similarly, in another aspect, the diagnostic system may derive the appropriate source volume of marrow needed based on CD34+ analysis. Much of the scientific literature and clinical literature for bone marrow cell therapy focuses on Mesenchymal Stem Cells (MSC) characterization and quantification (CFU-F assay). Historically, this is necessary to establish the treatment efficacy. CFU-F assay is costly, time consuming (up to 14 days to culture), and not clinically transferrable to the everyday treating physician. Thus, to date, there are no tools available to characterize and quantify MSCs rapidly in the point-of-care setting.

However, the total number of CD34+ cells from bone marrow (BM) and leukapheresis product (LKP) samples can be measured using, for example, an automatic cell counter or a direct flow cytometer. Determining the accurate absolute numbers of CD34+ cells is an important parameter for evaluating stem and progenitor cell content in hematopoietic transplantation. No device has been created to make this knowledge transferrable to the everyday treating physician utilizing bone marrow concentrate therapy. In various embodiments, the diagnostic treatment system and method utilizes the measured number of CD34+ cells from a sample to calculate the starting volume of blood or bone marrow a physician needs to collect in order to obtain the target volume of concentrate.

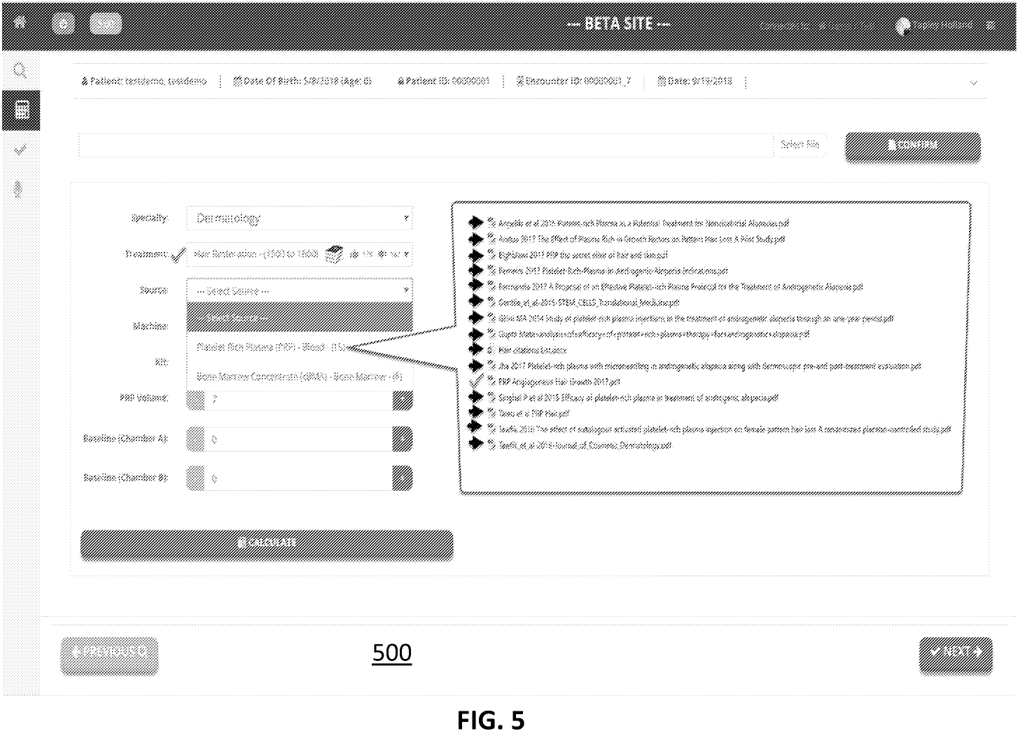

Referring now to FIG. 5, a screenshot 500 of an embodiment of the system is provided showing a software user interface displayed on a device. In some embodiments, a user may select specialty (e.g., Aesthetics/Medispa) from a menu or may input a specialty. The user may then select a treatment type (e.g., Hair Restoration) from a menu or may input a treatment type. In some embodiments, after the specialty information is inputted, the menu of treatment types may be limited to those related to the inputted specialty information. In some embodiments, the software may search a database of treatment protocols to provide a list of treatment types and the target range of cell concentrations for each treatment type. In other embodiments, a user may input a treatment type and the software will then search a database of treatment protocols to provide a target range of cell concentration for the inputted treatment types. In some embodiments, the user may input the target range of cell concentration or may increase or decrease the recommended target range. After the treatment type is inputted, the user may then select a source of the cells for the cell therapy, such as, for example, blood for a PRP treatment or bone marrow for a cBMA treatment. In some embodiments, a strength number may be shown next to the various sources available. For example, if a 15 was displayed next to PRP and a 6 was displayed next to cBMA, a user would be free to select either source, but would likely choose PRP as the source because of the higher strength number for PRP. In various embodiments, the strength number may be an indication of the number of research studies having positive treatment outcomes using that source for that treatment type. In some embodiments, summary information, such as title, publication, author, date, abstract, or other information may be provided to the user. In some embodiments, the user may select a research study to view additional information about that study. In some embodiments, once the user selects a particular study, the target cell concentration recommended in that study will be used in the calculation of the aspirate volume. The user may then enter information that will be used to calculate the recovery performance efficiency of the concentration device. In some embodiments, the user may input the type of centrifuge that will be used to concentrate the source fluid. In some embodiments, the user may input the kit that will be used to aspirate the source fluid from the patient. The user may then enter the volume of concentrate that will be used in the cell treatment therapy. In some embodiments, a volume to be used may be recommended based on the selected research study and the user may have the option to increase or decrease the volume. A user may input one or more baseline amounts for a particular user. In some embodiments, the baseline may be from a blood sample taken shortly before treatment, may be a patient’s historical baseline, or may be a default baseline based on demographic information of the patient. Using the inputted information, the software calculates a volume of source material needing to be aspirated. After the source material has been concentrated, a cell count may be taken and the results inputted back into the software (either automatically or manually entered). The software may then determine whether the actual concentration is within the target range. If the actual concentration is low, the system may recommend an additional amount to be aspirated. If the actual concentration is high, the system may recommend an amount of fluid (e.g., plasma, supernatant, or other liquid) to be added to dilute the concentration. In the screenshot 500, a timer is shown in the upper left corner of the display. In some embodiments, the timer may begin when the source fluid is aspirated and may alert the user when too much time has elapsed between aspiration and use. In some embodiments, the software may update the approximate concentration of the target solution based on the amount of time the timer has been running. For example, the software may lower the approximate concentration to account for cell decay due to the elapsed time.

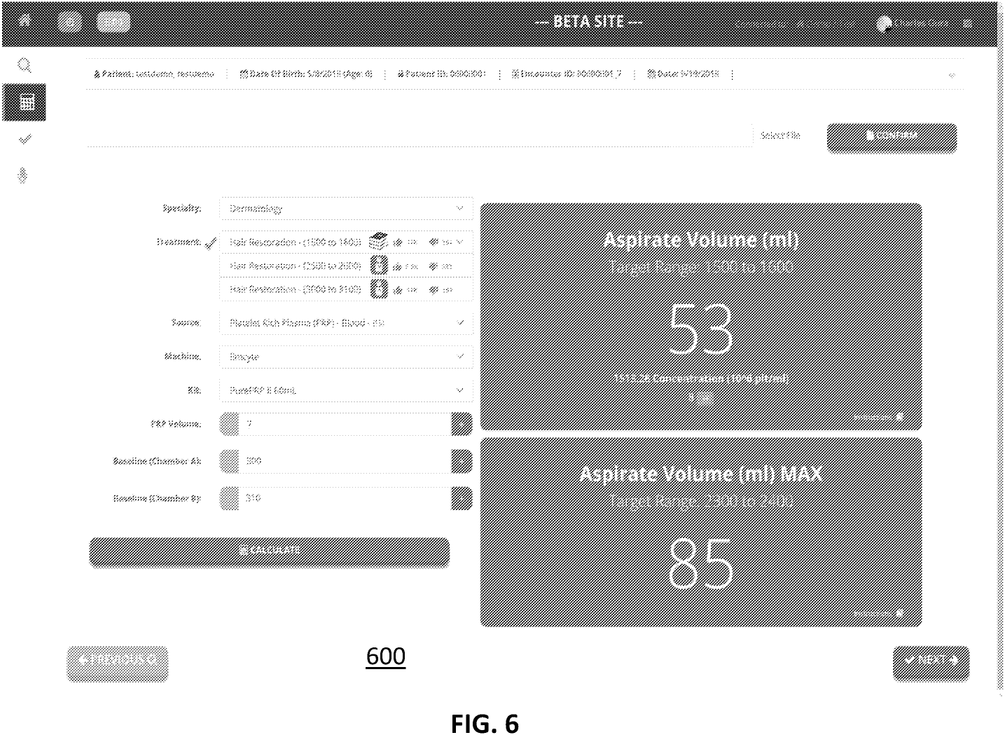

Referring now to FIG. 6, a screenshot 600 of an embodiment of the system is provided showing a software user interface displayed on a device. In the embodiment shown, in addition to a strength number being provided based on published research studies, a strength number is also provided based on the results of other users. In some embodiments, following treatment using a particular protocol, a user may provide an indication of the results obtained using that particular protocol. In the embodiment shown, the indication is displayed as a simple thumbs up or thumbs down along with a number. In other embodiments, a grade (e.g., A-F), a rating (e.g., on a scale of 1-5 or 1-10), a score (e.g., 70% success rate), or other indicia may be shown. In some embodiments, similar indicia may be provided for the other information to be inputted and/or selected by the user. In some embodiments, the indicia may be used in addition to or instead of indication of research studies. In some embodiments, the indicia based on user feedback may be correlated to one or more of the research studies.

Various systems and methods may simultaneously identify through published clinical literature the appropriate cell type, quantity of cells shown to be effective, and be agnostic to the specific concentration systems being used. Furthermore, the open architecture structure design allows the tool to be used compatibly with many commercially available cell counters. Combined with a cell counter, the diagnostic system contains embedded calculations to determine the appropriate source volume needed following a small aliquot sample prior to concentration. Following concentration, the diagnostic system can quantitatively verify the intended cell phenotype has been concentrated and calculate any additional volume that should be concentrated in order to provide information consistent with published literature prior to administering the cell therapy treatment.

In various embodiments, the diagnostic system may assist with therapies for treating, for example, Lateral Epicondylitis (i.e., tennis elbow); Medial Epicondylitis (i.e., golfer’s elbow); knee pain secondary to osteoarthritis (OA) and soft tissue injuries; hip pain secondary to OA and labral tears; Achilles tendonitis; Patellar Tendonitis (i.e., jumper’s knee); Plantar Fasciitis; OA - Thumbs; Rotator Cuff and Labral Tears of the Shoulder; Degenerative Disc Disease; Peri-operatively with bones grafts, Maxillofacial surgery dental implants; preoperatively and intraoperatively for total and partial joint replacements; soft tissue injuries such as meniscal, ligament and muscle tears, among others.

Although various embodiments of the method and apparatus of the present invention have been illustrated in the accompanying Drawings and described in the foregoing Detailed Description, it will be understood that the invention is not limited to the embodiments disclosed, but is capable of numerous rearrangements, modifications, and substitutions without departing from the spirit and scope of the invention.

What is claimed is:

1. A computer-implemented method for determining a volume of source material needed for a cell-therapy treatment comprising:
storing treatment protocols in a database, wherein the database is accessible to a computing device;
receiving a user selection of a cell-therapy treatment to be administered to a patient;
searching the database to identify treatment protocols related to the cell-therapy treatment;
receiving a user selection of a source material to be used in the cell-therapy treatment;
receiving a user selection of a cell concentration target range for the cell-therapy treatment, wherein the selected cell concentration target range corresponds to one or more of the identified treatment protocols;
receiving an input of a baseline cell count for the patient;
receiving an indication of a concentration device to be used to concentrate the source material, the concentration device having a recovery performance efficiency;
receiving a user indication of a treatment volume of concentrate to be used in the cell-therapy treatment;
calculating an aspiration volume of the source material to be aspirated for the cell-therapy treatment based on the selected cell concentration target range, the inputted baseline cell count for the patient, the recovery performance efficiency of the indicated concentration device, and the indicated treatment volume of concentrate; and
providing the aspiration volume of the source material to be displayed at the computing device.

2. The method of claim 1 and further comprising:
providing a strength number to be displayed on the computing device, the strength number being based at least in part on the identified treatment protocols.

3. The method of claim 1 and further comprising:
receiving a medical field of a physician that will be administering the cell-therapy treatment;
searching the database to identify a subset of the treatment protocols related to the medical field; and
providing a list of cell-therapy treatments corresponding to the subset of the treatment protocols.

4. The method of claim 3, wherein the receiving the user selection of the cell-therapy treatment to be administered to the patient comprises selecting the cell-therapy treatment from the list of cell-therapy treatments.

5. The method of claim 1 and further comprising:
receiving a cell count of cells in a sample of the concentrate;
calculating a cell concentration of the concentrate; and
determining whether the cell concentration of the concentrate is within the cell concentration target range.

6. The method of claim 5 and further comprising:
calculating an additional amount of source material to be aspirated if the cell concentration of the concentrate is below the cell concentration target range; and
calculating an amount of supernatant to add to the concentrate if the cell concentration of the concentrate is above the cell concentration target range.

7. The method of claim 6 and further comprising:
calculating an elapsed time between aspiration of the source material and injection of the concentrate into the patient; and
lowering the cell concentration of the concentrate if the elapsed time exceeds a predetermined threshold.

8. The method of claim 1 and further comprising calculating an elapsed time between aspiration of the source material and injection of the concentrate into the patient.

9. The method of claim 1 wherein the baseline cell count is determined based on a number of cells in a sample of the source material from the patient.

10. A computer readable medium for calculating a volume of source material needed for a cell-therapy treatment comprising:
a non-transitory computer readable medium having instructions stored thereon which when executed by one or more processors perform a process for calculating a volume of source material needed for a cell-therapy treatment, the process comprising:
receiving a user input of a cell-therapy treatment to be administered to a patient;
receiving a user input of a cell concentration target for the final concentrate to be used in the cell-therapy treatment;
receiving a user selection of a source material to be used in the cell-therapy treatment;
receiving a user input of a treatment volume of final concentrate to be used in the cell-therapy treatment;
receiving a user input of a baseline cell count for the patient;
receiving a user selection of a kit that will be used to aspirate the source material from the patient
receiving a user selection of a concentration device that will be used to concentrate the aspirated source material;
determining a recovery performance efficiency for the concentration device and the kit;
calculating an aspiration volume of the source material to be aspirated for the cell-therapy treatment based at least in part on (a) the cell concentration target, (b) the baseline cell count for the patient, (c) the recovery performance efficiency, and (d) the treatment volume of final concentrate; and
providing the aspiration volume of the source material to be displayed on a user device.

11. The computer readable medium of claim 10, wherein the process further comprises:
receiving feedback related to an outcome of the cell-therapy treatment, the feedback including an indication of whether the cell-therapy treatment outcome was positive.

12. The computer readable medium of claim 10, wherein the process further comprises:
receiving a cell count of cells in a sample of the final concentrate;
calculating a cell concentration of the final concentrate; and
determining whether the cell concentration of the final concentrate is within the cell concentration target.

13. The computer readable medium of claim 12, wherein the process further comprises:
calculating an additional amount of source material to be aspirated if the cell concentration of the final concentrate is below the cell concentration target; and
calculating an amount of supernatant to add to the final concentrate if the cell concentration of the final concentrate is above the cell concentration target.

14. The computer readable medium of claim 10, wherein the baseline cell count is determined based on a number of cells in a sample of the source material from the patient.

15. A diagnostic quality control system for cell-therapy treatments comprising:
a database for storing cell-therapy protocols, wherein each cell-therapy protocol provides a cell concentration target range;
a user device operatively connected to the database, the user device having computer readable instructions stored thereon which when executed by one or more processors perform a process for calculating a volume of source material needed for a cell-therapy treatment, the process comprising:
receiving a selection of a user specialty;
displaying cell-therapy treatments associated with the selected user specialty;
receiving a selection of a cell-therapy treatment from the cell-therapy treatments;
searching the database to identify cell-therapy protocols related to the cell-therapy treatment;
displaying the cell concentration target range for each identified cell-therapy protocol;
receiving a selection of a cell concentration target range of the displayed cell concentration target ranges;
receiving a selection of a source material to be aspirated;
receiving a selection of a concentration machine to be used to concentrate the aspirated source material;
receiving an input of a baseline cell count for the patient;
receiving an input of a treatment volume of final concentrate to be used in the cell-therapy treatment;
calculating a volume of source material to be aspirated based on the cell concentration target range, the concentration machine, the baseline cell count, and the treatment volume; and
displaying the volume of the source material on the user device.

16. The diagnostic quality control system of claim 15 and further comprising:
a cell counter operatively connected to the user device, the cell counter configured to receive a sample of the source material and send the baseline cell count for the patient to the user device.

17. The diagnostic quality control system of claim 15 and further comprising:
a cell counter operatively connected to the user device, the cell counter configured to receive a sample of the final concentrate and send a cell concentration of the final concentrate to the user device.

18. The diagnostic quality control system of claim 15, wherein the process further comprises:
receiving a cell count of cells in a sample of the final concentrate;
calculating a cell concentration of the final concentrate; and
determining whether the cell concentration of the final concentrate is within the cell concentration target range.

19. The diagnostic quality control system of claim 18, wherein the process further comprises:
calculating an additional amount of source material to be aspirated if the cell concentration of the final concentrate is below the cell concentration target range; and
calculating an amount of supernatant to add to the final concentrate if the cell concentration of the final concentrate is above the cell concentration target range.
